(19) United States
(12) Patent Application Publication
(10) Pub. No.: US 2012/0065532 A1
(43) Pub. Date: Mar. 15, 2012

(54) Implantable Medical Devices Employing Electroactive Polymers For Sensing Cardiac Motion and/or Causing Cardiac Compression

(75) Inventors: Kevin L. Morgan, Simi Valley, CA (US); Anne M. Shelchuk, Cupertino, CA (US); John R. Helland, Tallahassee, FL (US)

(73) Assignee: PACESETTER, INC., Sylmar, CA (US)

(21) Appl. No.: 12/882,095

(22) Filed: Sep. 14, 2010

(51) Int. Cl. A61B 5/0245; A61N 1/362

(52) U.S. Cl. 600/527; 607/9

(57) ABSTRACT

Disclosed herein is a system for monitoring a motion of a cardiac tissue. The system includes a motion sensor configured to operably couple to the cardiac tissue. The motion sensor includes an electroactive polymer and is further configured to result in a deflection in the electroactive polymer when the cardiac tissue undergoes the motion. The deflection in the electroactive polymer generates an electrical event.

### FIELD OF THE INVENTION

The present invention relates to medical devices and methods. More specifically, the present invention relates to devices and methods associated with sensing and causing cardiac function.

### BACKGROUND OF THE INVENTION

Congestive heart failure (CHF) or a myocardial infarction can result in heart tissue that is incapable of contributing to the pumping action of the heart. Where heart tissue damage is severe from the CHF or myocardial infarction, the affected heart tissue will even be non-responsive to pacing therapy provided via an implanted pulse generator such as, for example, a pacemaker or implantable cardioverter defibrillator (ICD).

Pacing and defibrillation therapies provided via an implantable pulse generator require the sensing of the natural cardiac electrical signals and the pulse generator administered electrical signals. For example, implantable medical leads extend from the pulse generator implanted in the patient's upper chest to locations within the patient's heart, for example, the right ventricle, right atrium and coronary sinus. The distal region of each lead includes electrodes for pacing and/or defibrillation. Also, the distal region of each lead further includes electrodes for sensing the natural cardiac and administered electrical signals. The pulse generator analyzes these sensed electrical signals to determine the how to administer the electrotherapy via the pacing and/or defibrillation electrodes.

Unfortunately, the sensing electrodes may sense electrical noise and far-field signals, which can mislead the analysis by the pulse generator. As a result, the pulse generator can administer inappropriate electrotherapy to the patient's heart, potentially leading to life threatening cardiac conditions.

There is a need in the art for systems, devices and methods for addressing the above-mentioned shortcomings.

### BRIEF SUMMARY OF THE INVENTION

Disclosed herein is a system for monitoring a motion of a cardiac tissue. In a first embodiment, the system includes a motion sensor configured to operably couple to the cardiac tissue. The motion sensor includes an electroactive polymer and is further configured to result in a deflection in the electroactive polymer when the cardiac tissue undergoes the motion. The deflection in the electroactive polymer generates an electrical event. The electrical event may include an electrical potential.

In a first version of the first embodiment of the system, the system may further include an implantable medical lead on which the motion sensor is supported, the lead being at least partially responsible for the motion sensor being operably coupled to the cardiac tissue. The lead may include a distal helical anchor configured to actively attach to the cardiac tissue, the anchor being at least partially responsible for the motion sensor being operably coupled to the cardiac tissue. The first version of the first embodiment of the system may yet further include a device electrically coupled to the motion sensor via the lead, the device configured to detect the electrical event and determine a characteristic of the cardiac motion from the electrical event. The characteristic may include heart rate, contractility, contractile velocity, or heart chamber contractile timing (i.e., A to V or V to V timing). The device may include an implantable pulse generator.

In a second version of the first embodiment of the system, the system may further include a substrate on which the motion sensor is supported, the substrate being at least partially responsible for the motion sensor being operably coupled to the cardiac tissue. The substrate may include a surface patch configured to be applied to the cardiac tissue. The surface patch may include at least one of an adhesive configured for cardiac tissue adherence or a mesh configured for cardiac tissue ingrowth. Alternatively or additionally, the surface patch may be at least one of configured to suture or staple to the cardiac tissue. The surface patch may have a shape that is generally rectangular, circular or cross-shaped. The second version of the first embodiment of the system may further include a lead and a device electrically coupled to the motion sensor via the lead, the device configured to detect the electrical event and determine a characteristic of the cardiac motion from the electrical event. The characteristic may include heart rate, contractility, contractile velocity, or heart chamber contractile timing (i.e., A to V or V to V timing). The device may include an implantable pulse generator.

In a third version of the first embodiment of the system, the system may further include a motion causing assembly configured to operably couple to the cardiac tissue and including an electroactive polymer that deflects upon being subjected to an electrical potential. The deflection of the electroactive polymer results in deflection of the motion causing assembly, thereby causing motion in the cardiac tissue operably coupled to the motion causing assembly. Thus, in some instances, the motion causing assembly may be considered a cardiac compression device, which when actuated, results in the compression of the region of the heart to which the motion causing assembly is operably coupled. The motion causing assembly may include a substrate on which the motion causing assembly is supported, the substrate being at least partially responsible for the motion causing assembly being operably coupled to the cardiac tissue. The substrate may include a surface patch configured to be applied to the cardiac tissue. The surface patch may include at least one of an adhesive configured for cardiac tissue adherence or a mesh configured for cardiac tissue ingrowth. Alternatively or additionally, the surface patch may be at least one of configured to suture or staple to the cardiac tissue. The third version of the first embodiment of the system may further include a lead and a device electrically coupled to the motion causing assembly via the lead, the device configured to generate the electrical potential. The device may include an implantable pulse generator.

Also disclosed herein is a method of monitoring a motion of a cardiac tissue. In a first embodiment, the method includes: operably coupling a motion sensor to the cardiac tissue in such a manner that motion of the cardiac tissue deflects an electroactive polymer of the motion sensor; and detecting an electrical event generated by deflection of the electroactive polymer. The electrical event may include an electrical potential. The first embodiment of the method may further include analyzing the detected electrical event to determine a characteristic of the cardiac motion from the electrical event. The characteristic may include heart rate, contractility, contractile velocity, or heart chamber contractile timing (i.e., A to V or V to V timing). The first embodiment of the method may additionally or alternatively include: operably coupling a motion causing assembly to the cardiac tissue; and subjecting an electroactive polymer of the motion causing assembly to an electrical potential, the deflection of the electroactive polymer resulting in deflection of the motion causing assembly, thereby causing motion in the cardiac tissue operably coupled to the motion causing assembly.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an anterior view of a heart shown in partial cross section with a cardiac motion sensing system coupled to the heart.

FIG. 2 is an isometric view of a section of a body of a lead depicted in FIG. 1, wherein an EAP sensor in a strip configuration is supported on the lead body.

FIG. 3 is an isometric view of a section of a body of a lead depicted in FIG. 1, wherein an EAP sensor in a semi-cylindrical configuration is supported on the lead body.

FIG. 4 is an isometric view of a section of a body of a lead depicted in FIG. 1, wherein an EAP sensor in a cylindrical configuration is supported on the lead body.

FIG. 5 is an anterior view of a heart with a another cardiac motion sensing system coupled to the heart.

FIG. 6 is an isometric view of a patch-shaped EAP sensor as may be employed according to FIG. 5.

FIG. 7 is an isometric view of a strip-shaped EAP sensor as may be employed according to FIG. 5.

FIG. 8 is an isometric view of a cross-shaped EAP sensor as may be employed according to FIG. 5.

FIG. 9 is a cross section elevation of an EAP sensor and a schematic wiring diagram extending therefrom.

FIG. 10 is an anterior view of a heart with a cardiac compression system coupled to the heart.

### DETAILED DESCRIPTION

Disclosed herein are medical devices employing electroactive polymer (EAP) to sense cardiac motion and/or cause cardiac compression. In one embodiment, an EAP equipped device can be coupled to heart tissue such that heart contractions will cause the EAP to flex, resulting in the generation of an electrical potential or charge that can be sensed and analyzed to determine cardiac operational characteristics such as, for example, heart rate and contractility. These cardiac operational characteristics can then be used to identify cardiac conditions such as, for example, ventricular tachycardia or ventricular fibrillation.

In another embodiment, an EAP equipped device can be coupled to heart tissue such that an electrical signal delivered to the EAP will cause the EAP equipped device to compress the heart tissue, causing a cardiac compression. Where such an EAP equipped device is coupled to heart tissue encompassing a myocardial infarction, the region of the myocardial infarction may be caused to contribute in the cardiac compression where traditional pacing-only therapies would fail to do so.

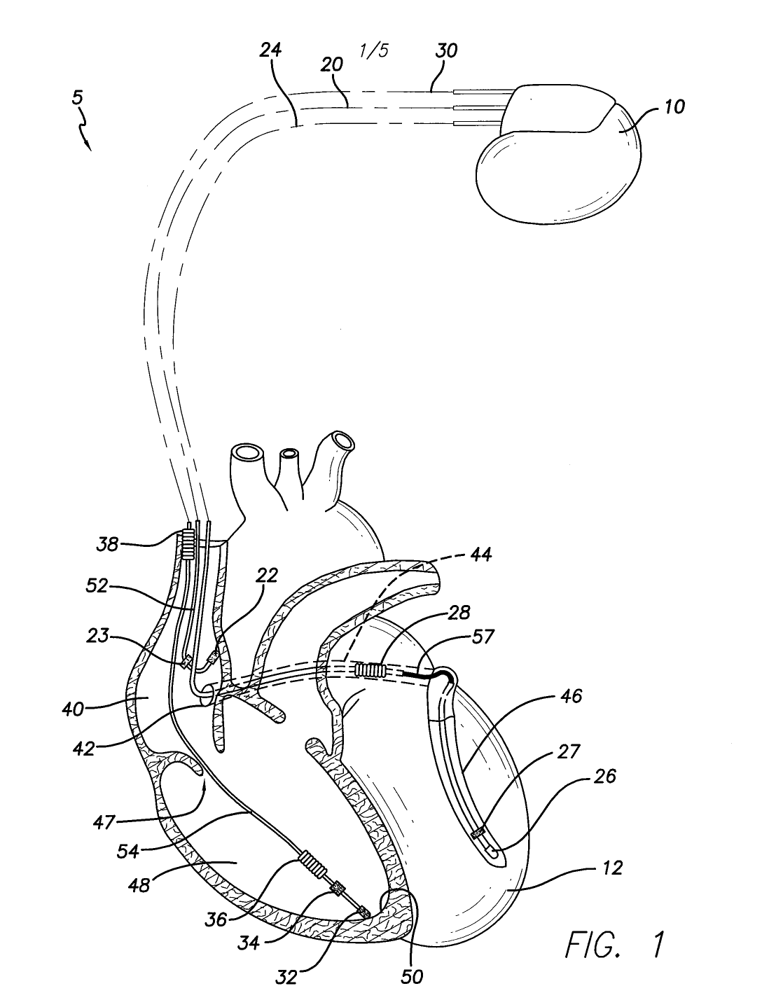

For a discussion of one embodiment of an EAP equipped device configured to sense cardiac motion, reference is made to FIG. 1, which is an anterior view of a heart 12 shown in partial cross section with a cardiac motion sensing system 5 coupled to the heart. As shown in FIG. 1, the system 5 includes an implantable pulse generator 10 such as, for example, a pacemaker or ICD and one or more EAP equipped implantable leads 20, 24, 30. A distal region of a lead may include one or more of the following items: a tip electrode 22, 26, 32, a ring electrode 23, 27, 34 proximal the tip electrode, and a defibrillation or shock coil 28, 36, 38 proximal the ring electrode.

A lead may be configured for passive fixation in the heart 12. In one embodiment, a distal region of a lead may be configured for passive fixation such as, for example, the lead body being configured to bias against the walls of the coronary sinus to passively maintain the lead in place. In one embodiment, the distal tip of a lead may be configured for passive fixation such as, for example, the lead distal tip having pliable tines radiating from the lead distal tip. In embodiments where the lead is configured for passive fixation, the tip electrode 22, 26, 32 may be in the form of a ring or semi-spherical dome.

The lead may be configured for active fixation. In one embodiment, a distal tip of a lead may be configured for active fixation, wherein the tip electrode 22, 26, 32 is in the form of a helical anchor electrode that allows the electrode to be screwed into cardiac tissue.

A proximal end of each lead 20, 24, 30 is coupled to the pulse generator 10 and the leads extend distally into the heart 12. For example, one lead 20 may extend from the pulse generator into the right atrium 40 where the lead tip electrode 22 is passively or actively fixed to a wall of the right atrium. Another lead 24 may extend from the pulse generator, through the right atrium, the coronary sinus ostium (OS) 42, and the coronary sinus 44, and into the left coronary vein 46. Yet another lead 30 may extend form the pulse generator, through the right atrium and tricuspid annulus 47 and into the right ventricle 48, the tip electrode 32 passively or actively fixed to the wall of the right ventricle near the right ventricular apex 50.

As indicated in FIG. 1, a lead 20, 24, 30 includes an EAP sensor 52, 54, 57 on the lead body near the lead distal end. The EAP sensor is located on a region of the lead body that is generally most likely to be deflected by cardiac motion when the lead distal end is fixed to a desired fixation location in the heart 12. For example, for lead 30, which has its lead distal end fixed to cardiac tissue near the right ventricular apex 50, the EAP sensor 54 is located on the lead body proximal the lead's three most distal electrodes 32, 34, 36, but still located in the right ventricle 48 at a location on the lead body that is likely to deflect when the right ventricle compresses. For another example, for lead 20, which has its lead distal end fixed to cardiac tissue in the right atrium 40 near the OS 42, the EAP sensor 52 is located on the lead body proximal the lead's electrodes 22, 23, but still located in the right atrium 40 at a location on the lead body that is likely to deflect when the right atrium compresses. For yet another example, for lead 24, which has its lead distal end fixed to cardiac tissue in the left coronary vein 46, the EAP sensor 57 is located on the lead body proximal the lead's two most distal electrodes 26, 27, but still located in the coronary sinus 44 or left coronary vein 46 at a location on the lead body that is likely to deflect when the left ventricle compresses.

Depending on the embodiment, an EAP equipped lead will have any one or more of the different types of the above-mentioned electrodes/coils and the EAP equipped lead will have one or more EAP sensors located on the lead body at any one or more locations that are generally likely to undergo the most deflection once the lead is implanted. An EAP sensor can be located at a variety of locations on an EAP equipped lead, and the examples discussed above are purely for illustrative purposes and not intended to limit the possibilities with respect to EAP sensor number or placement on a lead body. An EAP equipped lead with any of the electrode configurations discussed above may be employed with a variety of standard (i.e., non-EAP equipped) pacing leads in a variety of commonly employed lead placement strategies.

In some embodiments, an EAP equipped lead will have none of the above listed electrodes and will simply be a structure on which one or more EAP sensors may be mounted. Such an EAP sensor only type lead may be employed with a variety of standard (i.e., non-EAP equipped) pacing leads in a variety of commonly employed lead placement strategies.

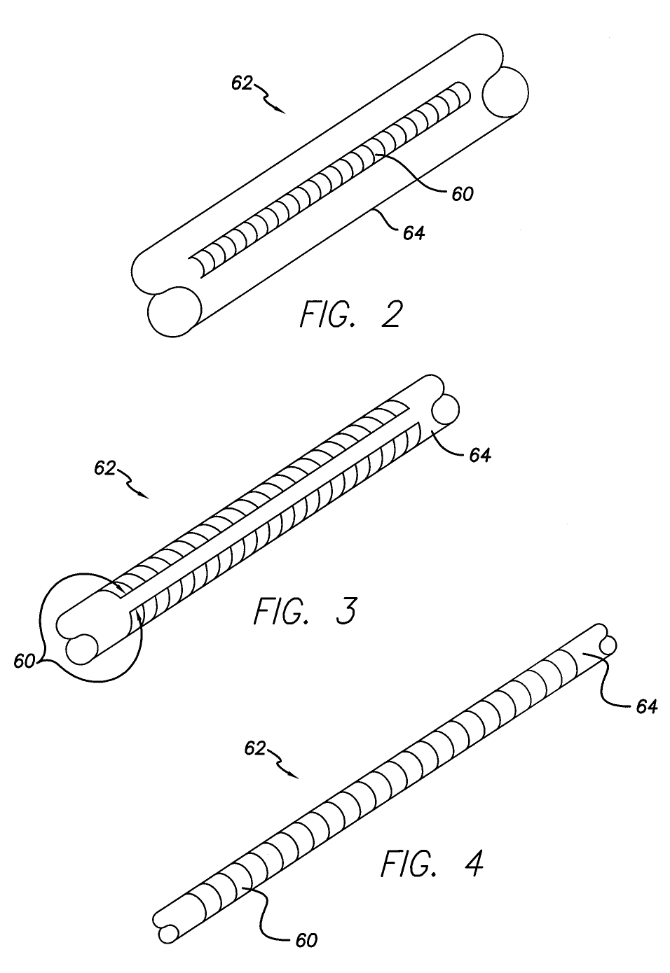

For a discussion of some example embodiments for lead based EAP sensors 60 that can be employed on lead bodies as discussed above with respect to FIG. 1, reference is made to FIGS. 2, 3 and 4, which are isometric views of a section of a lead body 62. As shown in FIG. 2, in one embodiment, an EAP sensor 60 may be in the form of a strip extending longitudinally along a length of the lead body 62. In one embodiment, such an EAP sensor 60 has a length of between approximately 5 mm and approximately 80 mm and a width that is between approximately 5 percent and 50 percent of the outer circumference of the lead body 62. While the EAP sensor 60 is illustrated in FIG. 2 as being on or forming part of the outer circumferential surface 64 of the lead body, in some embodiments, the EAP sensor 60 is imbedded in the lead body 62 below the outer circumferential surface 64. A segment length of a lead body 62 may have one or more such strip-type EAP sensors 60 located about the outer circumferential surface 64 of the lead body 62. For example, such strip-type EAP sensors 60 could be located at 12, 3, 6 and 9 o'clock about the outer circumference of the lead body.

As shown in FIGS. 3 and 4, in one embodiment, an EAP sensor 60 may be in the form of a partial or complete cylinder extending longitudinally along a length of the lead body 62. In one embodiment, such an EAP sensor 60 has a length of between approximately 5 mm and approximately 80 mm. As illustrated in FIG. 4, in one embodiment, the EAP sensor 60 is a complete cylinder extending uninterrupted about the outer circumference of the lead body. As depicted in FIG. 3, in one embodiment, the EAP sensor 60 is a semi-cylinder partially extending about the outer circumference of the lead body between approximately 10 percent and 50 percent of the outer circumference of the lead body 62. While the EAP sensor 60 is illustrated in FIGS. 3 and 4 as being on or forming part of the outer circumferential surface 64 of the lead body, in some embodiments, the EAP sensor 60 is imbedded in the lead body 62 below the outer circumferential surface 64.

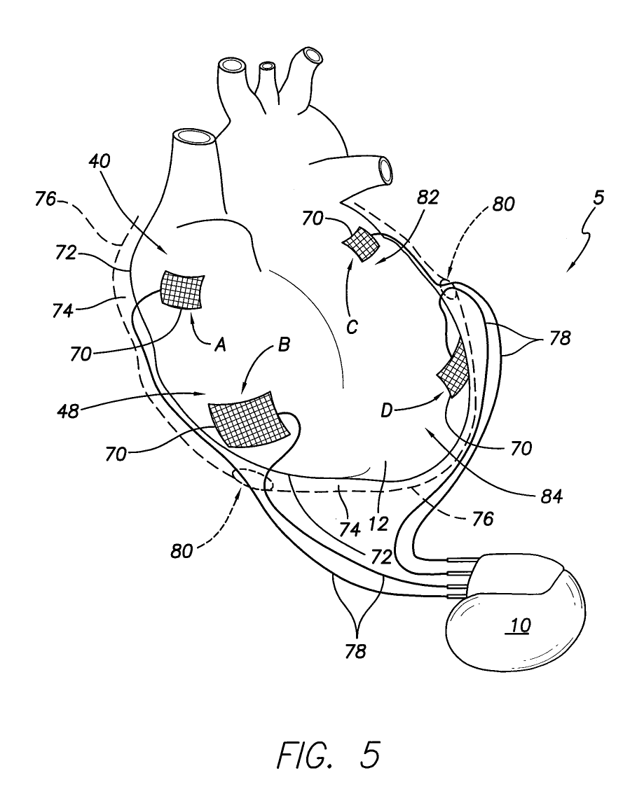

For a discussion of another embodiment of an EAP equipped device configured to sense cardiac motion, reference is made to FIG. 5, which is an anterior view of a heart 12 with a cardiac motion sensing system 5 coupled to the heart. As shown in FIG. 5, the system 5 includes an implantable pulse generator 10 and one or more EAP equipped implantable devices 70 configured for placement on the outer surface 72 of the heart 12. For example, in one embodiment, the EAP equipped surface mountable sensors 70 are implanted on the heart outer epicardial surface 72 in the pericardial space 74 defined between the heart outer epicardial surface 72 and the interior surface of the pericardial sac 76. A lead 78 having electrical conductors insulated therein extends proximally from each EAP equipped surface mountable device 70 to the pulse generator 10.

The devices 70 and leads 78 can be delivered minimally invasively via a subxyphoid access and punctures 80 in the pericardial sac 76. Each device 70 can be secured to the heart outer surface 72 via a variety of methods including, for example, adhesive, suture, screwed-in anchors, configurations that facilitate the device 70 being wedged into place to mechanically, forceably hold the device in place, and inflammatory response for causing tissue in-growth of cardiac tissue cells into a polyester mesh forming part of the device 70.

In some embodiments, the devices 70 are implanted on the outer surface of the pericardial sac 76. In some embodiments, the devices 70 include electrodes mounted thereon for providing pacing and/or defibrillation shocking.

As indicated in FIG. 5, the EAP equipped device 70 is located on a region of the outer surface 72 of the heart 12 where movement of the heart surface 72 is generally most likely to cause the device 70 to deform, displace or deflect in a manner that will cause the EAP of the device 70 to create an electrical signal that can be detected and analyzed by the pulse generator 10. For example, a device 70 may be mounted on the outer surface of the right atrium 40, as indicated at A. Alternatively or additionally, a device 70 may be mounted on the outer surface of the right ventricle 48, as indicated at B. Alternatively or additionally, a device 70 may be mounted on the outer surface of the left atrium 82, as indicated at C. Alternatively or additionally, a device 70 may be mounted on the outer surface of the left ventricle 84, as indicated at D. Of course, the device(s) 70 can be located on other regions of the heart outer surface 72, and the locations A-D are provided merely as non-limiting examples.

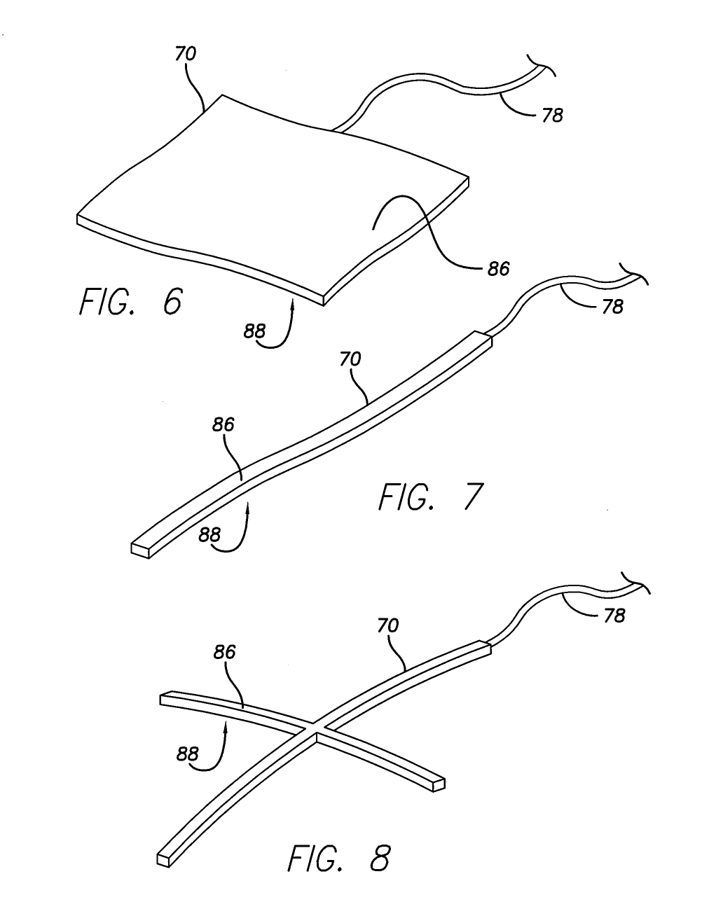

For a discussion of some example embodiments for EAP sensors 70 that are configured for mounting on a heart outer surface 72 as discussed above with respect to FIG. 5, reference is made to FIGS. 6, 7 and 8, which are isometric views of such sensors 70. As shown in FIG. 6, in one embodiment, an EAP sensor 70 may be in the form of a sheet or patch that is square, rectangular, triangular, circular, oval, or etc. and from which a lead 78 extends for coupling to the pulse generator 10. In a rectangular version of the EAP patch 70 of FIG. 6, the patch 70 has a length of between approximately 10 mm and approximately 40 mm and a width between approximately 5 mm and 25 mm.

As shown in FIG. 7, in one embodiment, an EAP sensor 70 may be in the form of a strip from which a lead 78 extends for coupling to the pulse generator 10. In one version of the EAP strip 70 of FIG. 7, the strip 70 has a length of between approximately 10 mm and approximately 50 mm and a width between approximately 1 mm and 15 mm.

As shown in FIG. 8, in one embodiment, an EAP sensor 70 may be in the form of a cross from which a lead 78 extends for coupling to the pulse generator 10. In one version of the EAP cross 70 of FIG. 8, the cross 70 arms have lengths from the cross intersection of between approximately 5 mm and approximately 40 mm, and the cross arms 70 have widths between approximately 2 mm and 20 mm. Of course, the configurations given in FIGS. 6, 7 and 8 are merely non-limiting examples, and other configurations are possible and should be considered as being part of the scope of this disclosure.

For each of the embodiments depicted in FIGS. 6, 7 and 8, the EAP sensor 70 includes an upper surface 86 and a bottom surface 88. The upper surface 86 includes or supports the EAP material, and the bottom surface 88 is configured to generally conform to the outer heart surface 72 when the EAP sensor is applied to the outer heart surface. In one embodiment, the bottom surface includes an adhesive for adhering the EAP sensor to the heart surface. In one embodiment, the bottom surface includes a polyester mesh or other material such as, for example, fibrin matrix or ePTFE, for causing inflammatory response that causes heart surface cells to in-grow into the mesh, thereby securing the EAP sensor 70 to the heart surface. In some embodiments, the EAP sensor is configured to be sutured, anchor screwed, wedged into place to mechanically, forceably hold the device in place, or otherwise mechanically anchored to the heart tissue.

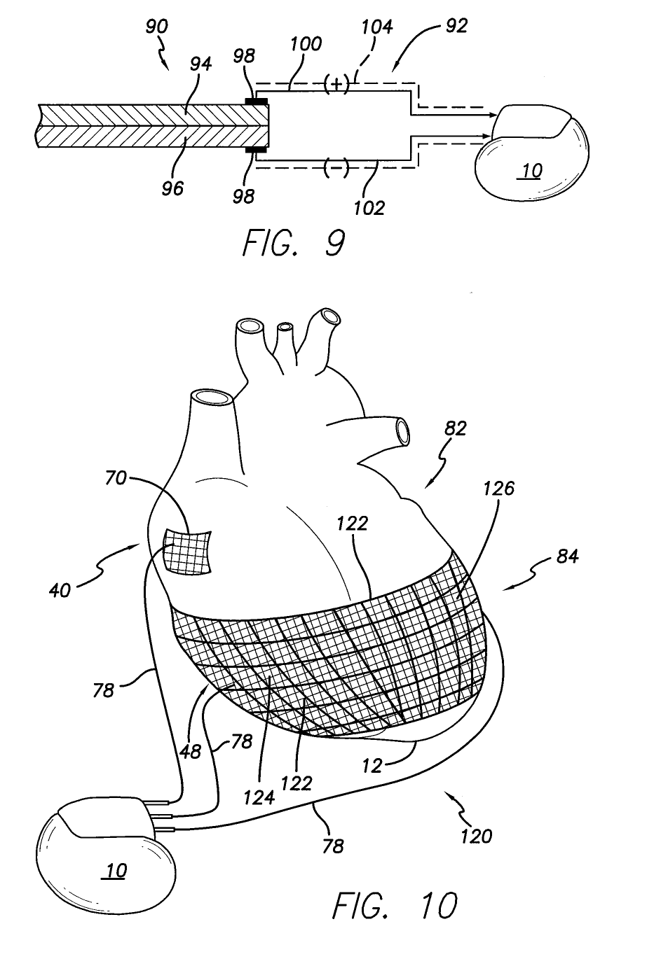

For a discussion of an EAP sensor configuration 90 that may be employed as part of any of the EAP sensors 60, 70 discussed above with respect to FIGS. 2-4 and 6-8, reference is made to FIG. 9, which is a cross section elevation of an EAP sensor configuration 90 and a schematic wiring diagram 92 extending therefrom. In one embodiment, as can be understood from FIG. 10, the EAP sensor configuration 90 employs an Ionomeric Polymer-Metal Composite (IPMC) 94 or another ionic EAP. A typical IPMC includes a thin (200 μm thick) polymer membrane 96 with metal electrodes 98 (5-10 μm thick) plated to both faces. The polyelectrolyte is neutralized with counter-ions, balancing the charge of the anions covalently fixed to the membrane 96. When an IPMC is hydrated and stimulated by a small voltage (1-5 V), both the fixed anions and the mobile counter-ions are subjected to the electric field. The counter-ions diffuse towards one of the electrodes and, as a result, the composite undergoes a fast bending deformation toward the anode. The bending is the result of increased stiffness along the cathode and decreased stiffness along the anode. Reversing the process, i.e., bending the IPMC instead of applying electricity to the IPMC, generates an electrical potential difference similar to electrical potential differences that arise in traditional piezoelectric-like behavior.

Current methods for IPMC manufacture rely on noble metals such as platinum and gold. Nafion® and Flemion® are common base polymers. The permselective properties of ionomeric resins allow selective reduction of metal salts at the surface of ion exchange membranes making polymer-metal composites that are not prone to delamination.

As indicated in FIG. 9, in one embodiment, a power conductor 100 and a ground conductor 102 extend from respective electrodes 98 of the EAP sensor configuration 90 within an electrically insulated lead body 104 to the pulse generator 10, as discussed above with respect to FIGS. 1 and 5. As can be understood from FIG. 9 and the preceding discussion regarding FIGS. 1-8, when the EAP sensor is secured to the heart tissue such that movement of the heart tissue cause a bending, flexing, deflection or any other deformation of the EAP configuration 90, an electrical potential difference is generated by the EAP configuration 90 that is transmitted to the pulse generator 10 via the conductors 100, 102 contained in the lead body 104. The pulse generator 10, or any other device electrically coupled to the EAP configuration and having algorithms for analyzing the EAP generated electrical potential difference, deciphers the electrical potential difference with respect to such example parameters as rate and magnitude. Such example parameters can be deciphered and monitored to determine such example cardiac characteristics as heart rate and contractility. In other words, the rate of the EAP flexion can be correlated with the heart rate, and the magnitude of the flexion can be correlated with contractility. These characteristics can be employed to diagnose cardiac conditions such as, for example, Ventricular tachycardia (VT) and Ventricular Fibrillation (VF).

In one embodiment, an EAP sensor as described above with respect to FIGS. 1-9 may be secured to a heart surface that is subject to regular flexion caused by the cardiac cycle. Thus, a variety of sensor locations are possible besides those indicated in FIGS. 1 and 5.

The wiring diagram 92 depicted in FIG. 9 illustrates a bipolar configuration. In some embodiments, wiring diagram 92 coupling the EAP configuration 90 to the pulse generator 10 may have a unipolar configuration.

The EAP sensors disclosed herein detect cardiac motion, which is used to determine heart rate, contractility, etc. Thus, unlike the sensing electrodes of leads, which relying on the sensing of electrical signals, the EAP sensors are less susceptible to electrical noise and far-field signal contributions than conventional electrical sensing.

For a discussion of one embodiment of an EAP equipped device configured to cause cardiac compression, reference is made to FIG. 10, which is an anterior view of a heart 12 with a cardiac compression system 120 coupled to the heart. As shown in FIG. 10, the system 120 includes an EAP compression device 122 and an implantable pulse generator 10 such as, for example, a pacemaker or ICD. The system may also include implantable leads similar to those discussed above with respect to FIG. 1 and/or conventional implantable leads. The system may also include EAP sensors 70 similar to those discussed above with respect to FIG. 5.

As can be understood from FIG. 10, the EAP compression device 122 extends about at least a portion of the outer surface of the heart, for example, in the pericardial space. The device 122 may be in the form of a pouch, harness or sack that envelops or encloses a substantial portion of the heart. For example, as shown in FIG. 10, the EAP compression harness 122 may extend around the right ventricle 48 and left ventricle 84. The compression harness 122 may be segmented. For example, the harness 122 may include a right portion 124 and a left portion 126 that are mechanically coupled together as part of the overall harness 122, but electrically isolated such that the right portion 124 and left portions 126 can be triggered separately to separately compress the ventricles 48, 84 as needed to achieve a mechanically actuated heart function that is similar to natural heart function. An electrical pacing signal sent to the compression harness 122 causes the EAP imbedded in the harness to flex, causing the harness 122 to artificially compress the heart regions enclosed by the harness 122. The pacing can be driven be the pulse generator 10 similar to conventional electrical pacing as known in the art.

Alternatively, the device 122 may be in the form of a patch or sheet that extends over a selected portion of the heart surface, but does not encompass or extend about the heart. For example, the sheet 122 may be secured over a region of the heart having an infarction, which benefits from both the reinforcement of the sheet 122 and the compression of the sheet as a pacing signal is delivered to the EAP imbedded in the sheet 122.

In some embodiments, EAP sensors on leads (as discussed with respect to FIGS. 1-5) and/or on patches or similar configurations (as discussed with respect to FIGS. 6-8) may be employed with the compression device 122 discussed with respect to FIG. 10. In such an embodiment, both the cardiac compression pacing and the compression sensing can be achieved without the electrical pacing and sensing common in the art.

Although the present invention has been described with reference to preferred embodiments, persons skilled in the art will recognize that changes may be made in form and detail without departing from the spirit and scope of the invention.

What is claimed is:

1. A system for monitoring a motion of a cardiac tissue, the system comprising:
a motion sensor configured to operably couple to the cardiac tissue and comprising an electroactive polymer, the motion sensor further configured to result in a deflection in the electroactive polymer when the cardiac tissue undergoes the motion, the deflection in the electroactive polymer generating an electrical event; and
an implantable medical lead on which the motion sensor is supported, the lead being at least partially responsible for the motion sensor being operably coupled to the cardiac tissue.

2. The system of claim 1, further comprising a substrate on which the motion sensor is supported, the substrate being at least partially responsible for the motion sensor being operably coupled to the cardiac tissue.

3. The system of claim 2, wherein the substrate includes a surface patch configured to be applied to the cardiac tissue.

4. The system of claim 3, wherein the surface patch comprises at least one of an adhesive configured for cardiac tissue adherence or a mesh configured for cardiac tissue ingrowth.

5. The system of claim 3, wherein the surface patch is at least one of configured to suture or staple to the cardiac tissue.

6. The system of claim 3, wherein the surface patch is configured to facilitate the surface patch being wedged into place to mechanically, forceably hold the surface patch against the cardiac tissue.

7. The system of claim 1, further comprising a device electrically coupled to the motion sensor via the lead, the device configured to detect the electrical event and determine a characteristic of the cardiac motion from the electrical event.

8. The system of claim 7, wherein the characteristic includes heart rate, contractility, contractile velocity, or heart chamber contractile timing.

9. The system of claim 1, further comprising a motion causing assembly configured to operably couple to the cardiac tissue and comprising an electroactive polymer that deflects upon being subjected to an electrical potential, the deflection of the electroactive polymer resulting in deflection of the motion causing assembly, thereby causing motion in the cardiac tissue operably coupled to the motion causing assembly.

10. The system of claim 9, wherein the motion causing assembly includes a substrate on which the motion causing assembly is supported, the substrate being at least partially responsible for the motion causing assembly being operably coupled to the cardiac tissue.

11. The system of claim 9, further comprising a lead and a device electrically coupled to the motion causing assembly via the lead, the device configured to generate the electrical potential.

12. A method of monitoring a motion of a cardiac tissue, the method comprising:
operably coupling a motion sensor to the cardiac tissue in such a manner that motion of the cardiac tissue deflects an electroactive polymer of the motion sensor; and
detecting an electrical event generated by deflection of the electroactive polymer.

13. The method of claim 12, wherein the electrical event includes an electrical potential.

14. The method of claim 12, further comprising analyzing the detected electrical event to determine a characteristic of the cardiac motion from the electrical event.

15. The method of claim 14, wherein the characteristic includes heart rate, contractility, contractile velocity, or heart chamber contractile timing.

16. The method of claim 14, further comprising:
operably coupling a motion causing assembly to the cardiac tissue; and
subjecting an electroactive polymer of the motion causing assembly to an electrical potential, the deflection of the electroactive polymer resulting in deflection of the motion causing assembly, thereby causing motion in the cardiac tissue operably coupled to the motion causing assembly.
